(19) United States
(12) Patent Application Publication
(10) Pub. No.: US 2026/0315545 A1
(43) Pub. Date: Oct. 15, 2026

(54) DYSOSMOBACTER FOR THE TREATMENT OF BREAST CANCER

(71) Applicant: UNIVERSITE CATHOLIQUE DE LOUVAIN, Louvain-la-Neuve (BE)

(72) Inventors: Patrice CANI, Bruxelles (BE); Bénédicte JORDAN, Sterrebeek (BE); Emilie MOENS DE HASE, Uccle (BE); Caner YELEK, Evere (BE)

(73) Assignee: UNIVERSITE CATHOLIQUE DE LOUVAIN, Louvain-la-Neuve (BE)

(21) Appl. No.: 19/101,374

(22) PCT Filed: Aug. 4, 2023
(86) PCT No.: PCT/EP2023/071660

§ 371 Date: Feb. 5, 2025

(30) Foreign Application Priority Data
Aug. 5, 2022 (EP) 22189007.2

(51) Int. Cl. A61K 35/74; A61K 35/00; A61P 35/00

(52) U.S. Cl. CPC A61K 35/74; A61P 35/00; A61K 2035/11

(57) ABSTRACT

A composition that includes bacteria from the genus Dysosmobacter, and/or variants, extracts or fragments thereof, and/or a culture supernatant of bacteria from the genus Dysosmobacter, and/or variants thereof. Also, a method for preventing and/or treating breast cancer in which the composition is administered in a therapeutically effective amount to a subject in need thereof.

### FIELD OF INVENTION

The present invention relates to the treatment and/or prevention of breast cancer using bacteria from the genus Dysosmobacter or extracts thereof.

### BACKGROUND OF INVENTION

Cancer has become one of the most important diseases of the last century and the leading cause of premature death on a global scale. It is estimated that roughly 19.3 million new cases of cancer occurred worldwide in 2020 while 10 million died from it. Among all cancers, breast cancer (BC) is now the most diagnosed cancer with an estimated 2.3 million cases representing 11.7% of all diagnoses and 25% of diagnosed women. However, breast cancer is a highly heterogeneous disease divided in subtypes based on the (over) expression of progesterone (PR), estrogen (ER) and human epidermal growth factor receptors (HER-2). Early and late-stage patients harboring hormone-receptor expressing breast tumors benefit from targeted therapies in combination or not with traditional chemotherapy as well as patients with HER-2 enriched tumors. However, patients with triple-negative breast cancer (TNBC), characterized by the absence of expression or overexpression of the previous receptors, do not benefit from targeted therapies. Because of these unmet needs in the field of targeted therapies, TNBC remains the most aggressive and with the poorest prognosis subtype associated with higher recurrence rate, higher metastatic potential and decreased survival. Risk factors for BC and TNBC comprise advanced age for first pregnancy, lack of physical activity and alcohol consumption, but obesity is increasingly seen as a major risk factor for BC and TNBC.

It has been described that obesity is linked with a higher risk of adverse outcome and poorer prognosis in breast cancer patients related with shorter overall and disease-free survival thus underlining the need to explore novel therapeutic strategies against breast cancer in an obese context. In obesity, important alterations of gut microbial composition, also called dysbiosis, occurs in clinics and preclinical models (Ley et al., 2006). Furthermore, the gut microbiota is a key regulator of whole-body metabolism and low-grade inflammation (Cani et al., 2019) but also gaining important focus in the modulation of cancer and breast cancer progression and therapy (Sampsell et al., 2020).

In the present invention, the inventors have investigated the impact of Dysosmobacter welbionis, a novel bacteria discovered, isolated, and named at UCLouvain (Brussels, Belgium) (Le Roy et al., 2020 and WO2020011856). Dysosmobacter welbionis is found in 70% of the general population and therefore can be considered as a highly prevalent bacterium, like Akkermansia muciniphila. As a short background on this bacterium, Dysosmobacter welbionis is a butyrate producer and Dysosmobacter spp and Dysosmobacter welbionis have been found to be less abundant in the intestine of subjects with obesity and type 2 diabetes. In preclinical studies, it has been shown that Dysosmobacter welbionis J115^{T} reduces body weight gain, fat mass gain and improves glucose tolerance, lipid and energy metabolism by acting on mitochondrial activity (Le Roy, Moens de Hase et al. 2022).

In the present disclosure, the inventors showed that Dysosmobacter welbionis J115^{T} could reduce breast cancer growth.

### SUMMARY

The present invention relates to a composition comprising at least (a) bacteria from the genus Dysosmobacter, and/or variants, and/or extracts and/or fragments thereof, and/or (b) a culture supernatant of bacteria from the genus Dysosmobacter and/or variants thereof, for use in preventing and/or treating breast cancer in a subject in need thereof. In some embodiments, the bacteria belong to the species Dysosmobacter welbionis.

In some embodiments, the bacteria belong to the strain J115^{T}, deposited at the BCCM/LMG on Mar. 14, 2018 as LMG P-30603.

In some embodiments, the breast cancer comprises at least one mutation on the gene encoding a receptor selected from the group comprising or consisting of Progesterone Receptor (PR), Estrogen Receptor (ER), and Human Epidermal Growth Factor Receptor-2 (HER2).

In some embodiments, the breast cancer is selected from the group comprising or consisting of luminal A breast cancer, luminal B breast cancer, HER2-positive breast cancer, and triple negative breast cancer (TNBC), preferably said breast cancer is triple negative breast cancer.

In some embodiments, the subject further suffers from one or more disease or condition that accelerates tumor growth selected from the group comprising or consisting of obesity-related disorders, liver diseases, metabolic disorders, adipokine-related disorders and inflammatory diseases, and combinations thereof.

In some embodiments, the subject further suffers from one or more disease or condition that accelerates tumor growth selected from the group comprising or consisting of obesity, non-alcoholic steatohepatitis (NASH), non-alcoholic fatty liver (NAFL), cirrhosis, diabetes mellitus, glucose intolerance, hyperglycemia, abnormal lipid metabolism, dyslipidemia, high cholesterol, elevated LDL-cholesterol, decreased HDL-cholesterol, elevated triglycerides and intestinal inflammation, and combinations thereof.

In some embodiments, the composition comprises a therapeutically effective amount of said bacteria, preferably from 1×10² to about 1×10¹⁵ CFU.

In some embodiments, the composition comprises live bacteria.

In some embodiments, the composition comprises dead or killed bacteria.

In some embodiments, the composition comprises pasteurized bacteria.

In some embodiments, the composition further comprises at least another anticancer agent.

In some embodiments, the composition is in the form of a pharmaceutical composition further comprising a pharmaceutically acceptable carrier.

The present invention further relates to a prebiotic comprising one or more active ingredients or substances that increase the level of bacteria from the genus Dysosmobacter in the microbiota of a subject in need thereof, for use in preventing and/or treating breast cancer in said subject.

The present invention further relates to a composition comprising at least one bacterium from the genus Dysosmobacter, and/or variants, extracts or fragments thereof, for use as an adjuvant to a treatment administered to a subject suffering from breast cancer.

### Definitions

In the present invention, the following terms have the following meanings:

“About” preceding a figure means plus or less 10% of the value of said figure.

“Acceptable”, for example when used in the expressions “Pharmaceutically acceptable” or “Nutraceutically acceptable” refers to molecular entities and compositions that do not produce an adverse, allergic or other untoward reaction when administered to a subject, especially a human, as appropriate.

“Bacterial strain” refers to a subtype of a bacterial species.

“Dysosmobacter” [Dys.os.mo.bac'ter. Gr. masc. adj. dysosmos bad smelling; N.L. masc. n. bacter a rod; N.L. masc. n. Dysosmobacter a bad-smelling rod] refers to a genus of bacteria described herein that have the following properties: cells are obligatory anaerobic, non-pigmented, non-spore-forming, non-motile, Gram-stain-negative. Cells form straight rods mainly 1.8-3.0 μm but often form elongated rods up to 20 μm whatever the growing phase. No respiratory menaquinones are produced. The genus belongs to the family of Ruminococcaceae. The type species is Dysosmobacter welbionis. In one embodiment, the diagnostic diamino acid in the cell wall is meso-2,6-diaminopimelic acid.

“Dysosmobacter welbionis” [wel.bi.o′nis. N.L. gen. n. welbionis] refers to a species of bacteria described herein that have the following properties in addition to the properties of the genus Dysosmobacter described hereinabove: colonies on solid modified YCFA after 72 h of incubation at 37° C. under anaerobic conditions are punctiform, cream, translucent, circular, entire, slightly convex and smooth. Growth is inhibited by the presence of 2% w/v bile or 2% w/v NaCl. Aesculin is not hydrolysed. Indole is not produced. Nitrate is not reduced. Gelatin is not digested. Urease is not produced. Catalase is not produced. Acid is produced from myo-inositol but not from D-glucose, D-arabinose, D-ribose and D-xylose. Positive reactions are obtained for arginine dihydrolase and glutamic acid decarboxylase. All the other tests from API 20A and Rapid ID 32A (bioMérieux, Lyon, France) are negative. Major fermentation end-products from myo-inositol are butyrate. The DNA GC content of the type strain is 59.3 mol % by High Performance Liquid Chromatography (HLPC). In one embodiment, the DNA GC content of the type strain is 58.9 mol % on the basis the genomic sequence. Type strain is J115^{T} (deposited at the Belgian Coordinated Collections of Microorganisms/Laboratory of Microbiology (BCCM/LMG), Universiteit Gent, K.L. Ledeganckstraat 35, 9000 Gent, Belgium, on Mar. 14, 2018 as LMG P-30603) and was isolated from human faeces. In one embodiment, the major cellular fatty acids are saturated branched-chain fatty acids and DMAs. In one embodiment, the major DMA fatty acid is C18:0 DMA and major saturated branched-chain fatty acids are iso-C15:0 and anteiso-C15:0.

“Fermentation” refers to the metabolic process that consumes sugar in the absence of oxygen. The products are organic acids, gases, or alcohol. It occurs in yeast and bacteria, and also in oxygen-starved muscle cells, as in the case of lactic acid fermentation.

“Gut microbiota” or “gastrointestinal microbiota” are used interchangeably to refer to the complex community of microorganisms that live in the digestive tracts of humans and other animals. The composition of the gastrointestinal microbiota changes over the lifetime of the host organism or when the diet of the host changes. It also varies across the digestive tract. The digestive tract contains a densely-populated microbial ecosystem with up to 10¹² cells per gram of intestinal content. Many species in the gut have not been studied outside of their hosts because most cannot be cultured. The four dominant bacterial phyla in the human gut are Firmicutes, Bacteroidetes, Actinobacteria, and Proteobacteria. Most bacteria belong to the genera Bacteroides, Clostridium, Faecalibacteria, Eubacteria, Ruminococcus, Peptococcus, Peptostreptococcus, Blautia, Subdoligranulum, Alistipes, Coprococcus, Dialister, Lachnoclostridium, Oscillospira, Parabacteroides, Prevotella, Roseburia, Ruminiclostridium, Sutterella and Bifidobacteria. Other genera, such as Escherichia, Enterococcus, Barnesiella, Butyricimonas, Butyricicoccus, Lachnospira, Odoribacter, Turicibacter and Lactobacillus, are present to a lesser extent. The gut microbiota is thought to function in the defense again pathogens, by competing with potential pathogens and by participating in the development of enteric protection and the immune system, metabolism, by assisting the digestion of consumed aliments, aiding the absorption of nutrients and synthetizing vitamins. The gut microbiota also interacts with the function of the central nervous system and the neuroendocrine and neuroimmune systems.

“Mutant”, as used herein, refers to a biological entity which has undergone a natural or induced (i.e. by mutagenesis) change in its genetic structure that does not interfere with the defining properties of said biological entity. The change in its genetic structure may be an insertion or deletion or substitution of one or several nucleotides in the genomic sequence. For example, a Dysosmobacter welbionis mutant refers to a Dysosmobacter welbionis strain which has undergone a change, natural or by techniques of genetic engineering, in its genetic structure that does not interfere with its belonging to the Dysosmobacter welbionis species.

“Nutraceutically effective amount” refers to the amount of a nutraceutical composition, food or dietary supplement or functional food necessary and sufficient for providing a physiological benefit or alleviating a discomfort in a subject.

“Pasteurized bacterium” refers to a bacterium submitted to a heat treatment (or heating process).

“Pharmaceutically acceptable carrier or excipient” refers to molecular entities and compositions that do not produce an adverse, allergic or other untoward reaction when administered to a subject, especially a human, as appropriate. It includes any and all solvents, dispersion media, coatings, antibacterial and antifungal agents, isotonic and absorption delaying agents and the like. For human administration, preparations should meet, pyrogenicity, general safety and purity standards as required by regulatory offices, such as FDA Office or EMA. A pharmaceutically acceptable carrier or excipient may thus refer to a non-toxic solid, semi-solid or liquid filler, diluent, encapsulating material or formulation auxiliary of any type.

“Prebiotic” refers to a substance, which may not be digested by a subject (for example, by humans), but which modulates composition and/or activity of the gut microbiota through its metabolization by microorganisms in the gut, thus conferring a beneficial physiological effect on the host.

“Probiotics” refers to microbial cell preparations (for example, living microbial cells) which, when administered in an effective amount, provide a beneficial effect on the health or well-being of a subject. By definition, all probiotics have a proven non-pathogenic character. In one embodiment, these health benefits are associated with improving the balance of human or animal microbiota in the gastro-intestinal tract, and/or restoring normal microbiota.

“Subject” refers to a warm-blooded animal, preferably a human, a pet or livestock. As used herein, the terms “pet” and “livestock” include, but are not limited to, dogs, cats, guinea pigs, rabbits, pigs, cattle, sheep, goats, horses and poultry. In some embodiments, the subject is a male or female subject, preferably a female subject. In some embodiments, the subject is an adult or a child. In some embodiments, the subject may be a “patient”, i.e., a subject who/which is awaiting the receipt of or is receiving medical care or was/is/will be the object of a medical procedure according to the methods of the present invention or is monitored for the development of a disease.

“Therapeutically effective amount” refers to the level or amount of an agent that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively, or additionally, the therapeutically effective amount may be administered after initiation of the disease, disorder, or condition, for a therapeutic action.

“Treatment” refers to both therapeutic treatment and prophylactic or preventative measures wherein the object is to prevent or slow down (lessen) the targeted pathologic condition or disorder, preferably wherein said condition is breast cancer. Those in need of treatment include those already with the disorder as well as those prone to have the disorder or those in whom the disorder is to be prevented. A subject or mammal is successfully “treated” if, after receiving a therapeutic amount of Dysosmobacter welbionis and/or a variant thereof and/or a fragment thereof according to the present invention the patient shows one or more of the following observable and/or measurable changes: amelioration related to one or more of the symptoms associated with the specific disease or condition, preferably breast cancer, reduction of morbidity and mortality and improvement in quality of life issues. The above parameters for assessing successful treatment and improvement in the disease are readily measurable by routine procedures familiar to a physician.

“Type strain” refers to as defined in the International Code of Nomenclature of Bacteria, as the nomenclatural type of the species and the reference point to which all other strains are compared to know whether they belong to that species. For example, strain J115^{T}, isolated from a faecal sample of a healthy 25 years old female, is the type strain of the species Dysosmobacter welbionis.

“Variant” refers to all the genetically or phenotypically distinct strains of a species that retain the species-defining characteristics. The term variant is also used in reference to other phylogenetic taxa such as for a genus or for a strain. As used herein, the term “variant” refers to both naturally occurring and specifically developed variants or mutants of the bacteria disclosed and exemplified herein. In one embodiment, variants may or may not have the same identifying biological characteristics of the bacteria exemplified herein, provided they share similar advantageous properties in terms of treating or preventing diseases. In one embodiment, a variant of the bacteria of the invention has the same functional and/or therapeutic properties as the bacteria of the invention. Illustrative examples of suitable methods for preparing variants of the microbial strains exemplified herein include, but are not limited to, gene integration techniques such as those mediated by insertion of elements or transposons or by homologous recombination, other recombinant DNA techniques for modifying, inserting, deleting, activating or silencing genes, intraspecific protoplast fusion, mutagenesis by irradiation with ultraviolet light or X-rays, or by treatment with a chemical mutagen such as nitrosoguanidine, methyl methane sulfonate, nitrogen mustard and the like, and bacteriophage-mediated transduction.

### DETAILED DESCRIPTION

This invention relates to a composition comprising at least (a) bacteria from the genus Dysosmobacter, and/or variants, extracts or fragments thereof, and/or (b) a culture supernatant of bacteria from the genus Dysosmobacter, and/or variants thereof, for use in preventing and/or treating breast cancer in a subject in need thereof.

In some embodiments, bacteria from the genus Dysosmobacter comprised in the composition for use according to the invention belong to the species selected from the group comprising or consisting of Dysosmobacter welbionis, Dysosmobacter acutus, Dysosmobacter segnis, and Dysosmobacter hominis. In some embodiments, bacteria from the genus Dysosmobacter comprised in the composition for use according to the invention belong to the species selected from the group comprising or consisting of Dysosmobacter welbionis.

Biological characteristics of Dysosmobacter welbionis have been reported previously by the inventors in WO2020011856.

In some embodiments, the bacteria belong to the strain selected from the group comprising or consisting of J115^{T}, Dysosmobacter acutus MSJ-2^{T} (CGMCC Accession number: 1.32896T; KCTC Accession number: 15976T), Dysosmobacter segnis BX15 (CGMCC Accession number: 1.32894), Marseille-Q4140, MM13, and Dysosmobacter hominis NSJ-60 (CGMCC Accession number: 1.32836; KCTC Accession number: 25148). In some embodiments, the bacteria belong to the strain J115^{T}, deposited at the BCCM/LMG on Mar. 14, 2018 as LMG P-30603, and/or a variant thereof. The J115^{T} strain is the type strain of the species Dysosmobacter welbionis. In a particular aspect, the invention relates to a composition comprising bacteria from the strain Dysosmobacter welbionis J115^{T} (deposit number LMG P-30603), and/or variants, extracts or fragments thereof, for use in preventing and/or treating breast cancer in a subject in need thereof.

In one embodiment, the genome sequence of the bacteria has the sequence SEQ ID NO: 1, or has a sequence presenting at least about 65% identity with SEQ ID NO: 1, preferably at least about 70%, 75%, 80%, 85%, 90% identity with SEQ ID NO: 1, more preferably at least about 91%, 92%, 93%, 94%, 95%, 96%, 96.5%, 97%, 97.5%, 97.6%, 97.7%, 97.8%, 97.9%, 98.0%, 98.1%, 98.2%, 98.3%, 98.4%, 98.5%, 98.6%, 98.65%, 98.7%, 98.8%, 98.9%, 99%, 99.1%, 99.2%, 99.3%, 99.4%, 99.5%, 99.6%, 99.7%, 99.8%, 99.9% or more identity with SEQ ID NO: 1.

In one embodiment, the bacteria have an Average Nucleotide Identity (ANI) score above about 60, preferably above about 74, 75, 80, 85, 90, more preferably above about 95, even more preferably above about 96, 97, 98, 98.5, 98.65, 99 or more when compared to the genome of sequence SEQ ID NO: 1.

Techniques to determine the ANI value are known to the person skilled in the art (such as methods implemented in Kim et al., Int J Syst Evol Microbiol. 2014 February; 64 (Pt 2): 346-51). Briefly, ANI correspond to corresponds to the sum for each bidirectional best hit (BBH-orthologs sequences identified on the basis of their position in the genome and sequence identity) of the identity multiplied by the length of the alignment divided by the total length of BBH genes.

In one embodiment, the bacteria have a hybrid DNA-DNA hybridization value (also referred to as DDH value) with SEQ ID NO: 1 above about 60%, preferably above about 61%, 62%, 63%, 64%, 65%, 66%, 67%, 68%, 69%, more preferably above about 70%.

Techniques to determine a DDH value are known to the person skilled in the art (such as methods reviewed in Stackebrandt et al., Molecular Identification, Systematics, and Population Structure of Prokaryotes, p23-50, 2006, Springer, Berlin, Heidelberg) and rely on the following general principle: (i) shearing the genomic DNA (gDNA) of the assayed organism and the gDNA of the reference organism(s) (for instance in the context of the present invention the type strain J115^{T} (deposited at the BCCM/LMG on Mar. 14, 2018 as LMG P-30603) into small fragments of 600-800 bp; (ii) heating the mixture of DNA fragments from both strains to dissociate the DNA double-strands; and (iii) subsequently decreasing the temperature until the fragments reanneal. For the reason that the melting temperature of a double-strand depends on the degree of matching base pairings between both strands, genomic (dis-) similarity can be inferred from the melting temperature. The hybrid DDH value is usually specified relative to the DDH value obtained by hybridizing a reference genome with itself. DDH values≤70% may be considered as an indication that the tested organism belongs to a different species than the type strain used as reference. The DDH value may also evaluated on the basis of the genomic sequence of the strains to be compared using in publicly available computer programs.

In one embodiment, the bacteria have an intergenome distance with SEQ ID NO: 1, below about 0.5, preferably below about 0.22, 0.21, 0.20, 0.19, 0.18, 0.17, 0.16, 0.15,0.14 more preferably bellow about 0.13, 0.12, 0.11, 0.10, or less.

Techniques to determine the intergenome distance, or genome-genome distance (GGD), are known to the skilled artisan. For example, methods described by Meier-Kolthoff et al. (BMC Bioinformatics 2013; 21:14-60; Int J Syst Evol Microbiol 2014; 1:352-6) may be used. Such method may be implemented using the genome calculator 2.1 (Deutsche Sammlung von Mikroorganismen und Zellkulturen—DSMZ) using BLAST+ as a local alignment tool and the sum of all identities found in high-scoring segment pairs (HSP) divided by overall HSP length.

In one embodiment, the nucleotide sequence of the 16S rRNA gene of the bacteria has the sequence SEQ ID NO: 2, or has a sequence presenting at least about 90% identity with SEQ ID NO: 2, preferably at least about 91%, 92%, 93%, 94%, 95%, 95.5%, 96%, 96.5%, 97%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.65%, 98.7%, 98.8%, 98.9%, 99%, 99.1%, 99.2%, 99.3%, 99.4%, 99.5%, 99.6%, 99.7%, 99.8%, 99.9% or more identity with SEQ ID NO: 2 (deposited under the GenBank/EMBL/DDBJ accession number MG963288).

In one embodiment, the nucleotide sequence of the 16S rRNA gene of the bacteria has the sequence SEQ ID NO: 2, or has a sequence presenting at least about 99.9% identity with SEQ ID NO: 2, preferably at least about 99.91%, 99.92%, 99.93%, 99.94%, 99.95%, 99.96%, 99.97%, 99.98%, 99.99%, or more identity with SEQ ID NO: 2.

In one embodiment, the nucleotide sequence of the 16S rRNA gene of the bacteria has the sequence SEQ ID NO: 2, or has a sequence presenting at least about 90% identity or more identity over the entire length of SEQ ID NO: 2, preferably at least about 91%, 92%, 93%, 94%, 95%, 96%, 96.5%, 97%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.65%, 98.7%, 98.8%, 98.9%, 99%, 99.1%, 99.2%, 99.3%, 99.4%, 99.5%, 99.6%, 99.7%, 99.8%, 99.9%, 99.91%, 99.92%, 99.93%, 99.94%, 99.95%, 99.96%, 99.97%, 99.98%, 99.99%, or more identity over the entire length of SEQ ID NO: 2.

The term “identity” when used in a relationship between the sequences of two or more polypeptides or of two or more nucleic acid molecules, refers to the degree of sequence relatedness between polypeptides or nucleic acid molecules, as determined by the number of matches between strings of two or more amino acid or nucleotide residues. “Identity” measures the percent of identical matches between the smaller of two or more sequences with gap alignments (if any) addressed by a particular mathematical model or computer program (i.e., “algorithms”). Identity of related polypeptides can be readily calculated by known methods.

The composition for use according to the invention may comprise variants of the bacteria as described hereinabove. Said variant may also be referred as a derived strain of the bacteria. In one embodiment, the variant of the bacteria may be obtained by mutation, variation or recombination of the bacteria described herein. Within the scope of the present invention, a variant may also be referred to as a mutant.

In one embodiment, the variant of the bacteria from the genus Dysosmobacter comprised in the composition for use according to the invention is a variant of Dysosmobacter welbionis. In one embodiment, the variant of the bacteria from the genus Dysosmobacter comprised in the composition for use according to the invention is a variant of Dysosmobacter welbionis strain J115^{T}.

In one embodiment, the variant of the bacteria has a genome at least about 70%, preferably at least about 80%, at least about 90%, at least about 91%, 92%, 93%, 94%, 95%, 96%, 97%, 98%, 99%, 99.1%, 99.2%, 99.3%, 99.4%, 99.5%, 99.6%, 99.7%, 99.8%, 99.9, or more identical to the genome of the bacteria from which it derives.

In one embodiment, the genome sequence of the variant of the bacteria has at least about 65% identity with sequence of the genome of the bacterium from which it derives, preferably at least about 70%, 75%, 80%, 85%, 90% identity with sequence of the genome of the bacterium from which it derives, more preferably at least about 91%, 92%, 93%, 94%, 95%, 96%, 96.5%, 97%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.65%, 98.7%, 98.8%, 98.9%, 99%, 99.1%, 99.2%, 99.3%, 99.4%, 99.5%, 99.6%, 99.7%, 99.8%, 99.9%, or more, identity with the sequence of the genome of the bacteria from which it derives.

In one embodiment, the variant of the bacteria has the same function and/or therapeutic properties as the bacterium from which it derives.

In some embodiments, the bacteria from the genus Dysosmobacter and/or variant thereof comprised in the composition for use according to the invention are live bacteria or killed bacteria.

Within the scope of the present invention, the term “live bacteria” is interchangeably used with the term “viable bacteria” and refers to bacteria that are able to proliferate in opposition to “killed bacteria” (or “non-viable bacteria”) that are not able to proliferate. Methods for measuring viability and proliferation are known to one skilled in the art. For example, bacteria viability and proliferation may be assessed by spreading a solution containing at least one bacterium of the invention across a petri dish and counting the number of colonies after a determined time of incubation in optimal growth conditions. Alternatively, bacteria may be grown in liquid medium, and proliferation may be measured by measuring optical density of the bacterial culture after a determined time of incubation in optimal growth conditions. It is also possible to determine the number of bacteria, including viable as well as non-viable bacteria by microscopic observation. While phase-contrast microscopy is a well-known method to do so, the microbial bacteria can be further visualized by specific staining with dyes, fluorescent probes or antibodies to facilitate microscopic observations or count bacteria by flow cytometry.

In some embodiments, the bacteria from the genus Dysosmobacter and/or variant thereof comprised in the composition for use according to the invention are live bacteria. In some embodiments, the bacteria are metabolically active.

In some embodiments, the bacteria from the genus Dysosmobacter and/or variant thereof comprised in the composition for use according to the invention are killed bacteria. In some embodiments, the bacteria and/or variant thereof are unable to proliferate. In some embodiments, the bacteria and/or variant thereof are metabolically inactive. In some embodiments, the bacteria and/or variant thereof are heat-inactivated or heat-killed.

Heat-inactivated or heat-killed bacterium can be obtained by heating at a temperature of at least about 90° C., preferably at least about 100° C., 105° C., 110° C., 115° C. or 120° C., more preferably at least about 121° C., 125° C., 130° C., 135° C., 140° C. or more. Heating may be performed for at least about 5 minutes, preferably for at least about 10 minutes, 15 minutes, 20 minutes, 25 minutes, 30 minutes or more. Heating may be performed using a saturating steam pressure of at least about 10 psig, preferably at least about 11, 12, 13, 14, 15 or more psig.

In some embodiments, the bacteria from the genus Dysosmobacter and/or variant thereof comprised in the composition for use according to the invention are pasteurized bacteria.

In one embodiment, the pasteurized bacteria and/or variant thereof were heated at a temperature ranging from about 50° C. to about 100° C., preferably from about 60° C. to about 95° C., more preferably from about 70° C. to about 90° C. In one embodiment, the pasteurized bacteria and/or variant thereof were heated at a temperature of about 50, 51, 52, 53, 54, 55, 56, 57, 58 or 59° C. In another embodiment, the pasteurized bacteria and/or variant thereof were heated at a temperature of about 60, 61, 62, 63, 64, 65, 66, 67, 68 or 69° C. In yet another embodiment, the pasteurized bacteria and/or variant thereof were heated at a temperature of about 70, 71, 72, 73, 74, 75, 76, 77, 78 or 79° C. In yet another embodiment, the pasteurized bacteria and/or variant thereof were heated at a temperature of about 80, 81, 82, 83, 84, 85, 86, 87, 88 or 89° C. In yet another embodiment, the pasteurized bacteria and/or variant thereof were heated at a temperature of about 90, 91, 92, 93, 94, 95, 96, 97, 98, 99° C. or 100° C.

In one embodiment, the pasteurized bacteria and/or variant thereof were not heated at a temperature superior to about 100° C. In a particular embodiment, the pasteurized bacteria and/or variant thereof were not heated at an ultra-high temperature, for example at a temperature ranging from about 110° C. to about 140° C. In one embodiment, the pasteurized bacteria and/or variant thereof were not heated at a temperature superior to about 90° C. Accordingly, in one embodiment of the invention, the bacteria and/or variant thereof were not sterilized. Sterilization is a treatment intended to destroy, kill or inactivate all life forms and other biological agents. This includes microorganisms and their spores as well as viruses and prions. Unlike sterilization, pasteurization is not intended to kill all microorganisms but is usually applied to food with the aim to reduce the number of viable pathogens.

In one embodiment of the invention, the pasteurized bacteria and/or variant thereof were heated for at least about 10 minutes. In another embodiment of the invention, the pasteurized bacteria and/or variant thereof were heated for at least about 15, 20, 25, 30, 35 or 45 minutes. In one embodiment, the pasteurized bacteria and/or variant thereof were heated for a period from about 10 to about 45 minutes.

In one embodiment, the pasteurized bacteria and/or variant thereof were not heated for a short time. In a particular embodiment, the pasteurized bacteria and/or variant thereof were not heated for less than about 30 seconds, less than about 60 seconds, less than about 90 seconds or less than about 120 seconds. In a preferred embodiment, the pasteurized bacteria and/or variant thereof were not heated for a time of less than about 1 minute, preferably for a time of less than about 5, 6, 7, 8, or 9 minutes.

In one embodiment, the pasteurized bacteria and/or variant thereof were heated at a temperature ranging from about 50° C. to about 100° C. for at least about 10 minutes. In a particular embodiment, the pasteurized bacteria and/or variant thereof were heated to about 60° C. for about 20 or about 30 minutes. In another particular embodiment, the pasteurized bacteria and/or variant thereof were heated to about 70° C. for about 20 or about 30 minutes. In another particular embodiment, the pasteurized bacteria and/or variant thereof were heated to about 80° C. for about 20 or about 30 minutes. In another particular embodiment, the pasteurized bacteria and/or variant thereof were heated to about 90° C. for about 20 or about 30 minutes.

In a particular embodiment, the pasteurized bacteria and/or variant thereof were not heated at a temperature superior to about 110° C. for about 1 to about 120 seconds. In another particular embodiment, the pasteurized bacteria and/or variant thereof were not heated at a temperature superior to about 100° C. for about 1 to about 120 seconds. In another particular embodiment, the pasteurized bacteria and/or variant thereof were not heated at a temperature superior to about 90° C. for about 1 to about 120 seconds.

In one embodiment, the bacteria and/or variant thereof are treated with an ultra-high temperature (UHT) treatment.

As used herein, a “UHT” treatment refers to an Ultra-high temperature processing or an ultra-heat treatment (both abbreviated UHT) involving the at least partial sterilization of a composition by heating it for a short time, such as from about 1 to about 60 seconds, preferably from about 1 to about 30 seconds, more preferably from about 1 to about 10 seconds, at a temperature of at least about 135° C.

There are two main types of UHT systems: the direct and indirect systems. In the direct system, products are treated by steam injection or steam infusion, whereas in the indirect system, products are heat treated using plate heat exchanger, tubular heat exchanger or scraped surface heat exchanger. Combinations of UHT systems may be applied at any step or at multiple steps in the process of product preparation.

In one embodiment, the bacteria and/or variant thereof are flash pasteurized. Accordingly, in one embodiment, the bacteria and/or variant thereof are treated at a temperature ranging from about 71.5° C. to about 74° C. for a period of time ranging from about 15 to about 30 seconds.

In some embodiments, the bacteria from the genus Dysosmobacter and/or variant thereof comprised in the composition for use according to the invention are fresh. The term “fresh” as used herein mean that the bacteria were not frozen between the last amplification phase and the use.

In some embodiments, the bacteria from the genus Dysosmobacter and/or variant thereof comprised in the composition for use according to the invention are not fresh. In some embodiments, the bacteria and/or variant thereof were frozen at least once. In some embodiments, the bacteria and/or variant thereof are frozen.

As used herein the term ‘frozen’, refers to a bacterium that is cooled down at or below a temperature allowing a phase transition from liquid to solid in said bacterium. In one embodiment said temperature is about −5°, −20° C., −70° C., −80° C. or −190° C.

In one embodiment, recovered frozen bacteria and/or variant thereof are viable. In other words, in one embodiment, the bacteria from the genus Dysosmobacter and/or variant thereof comprised in the composition for use according to the invention are frozen and viable.

In some embodiments, the composition for use according to the invention comprises fragments of bacteria from the genus Dysosmobacter, preferably fragments of Dysosmobacter welbionis, more preferably fragments of Dysosmobacter welbionis strain J115^{T}.

As used herein, the term “fragment” refers to bacterial components, metabolites, secreted molecules and/or vesicles and compounds resulting from the metabolism of the bacteria and/or variant thereof comprised in the composition for use according to the invention, and the like. Examples of bacterial components include, but are not limited to, bacterial cell wall components such as peptidoglycan, bacterial nucleic acids such as DNA and RNA, bacterial membrane components, and bacterial structural components such as proteins, carbohydrates, lipids and combinations of these such as lipoproteins, glycolipids and glycoproteins, bacterial metabolites, organic acids, inorganic acids, bases, peptides, enzymes and co-enzymes, amino acids, carbohydrates, lipids, glycoproteins, lipoproteins, glycolipids, vitamins, bioactive compounds and metabolites containing an inorganic component. Fragments may be obtained by recovering the supernatant of a culture of the bacteria and/or variant thereof or by extracting cell components or cell fractions, metabolites or secreted compounds from a culture of the bacteria and/or variant thereof, a degradation product, a component in the isolated form, any mixture of one or more components derived from the bacteria and/or variant thereof, or one or more components present in the bacteria and/or variant thereof that are produced in another way, for example using recombinant DNA technology, in a microbial host or in any other (bio) synthetic process. In one embodiment, the fragments are comprised in the culture medium of the bacteria, i.e., the fragments are not purified nor isolated. In another embodiment, the fragments are purified by methods known in the art, e.g., affinity purification.

In some embodiments, the composition for use according to the invention comprises an extract of the supernatant of the culture medium of the bacteria from the genus Dysosmobacter, and/or variant thereof.

Culture medium suitable for bacterial culture are known in the art. In some embodiments, and in particular for the culture of bacteria from the genus Dysosmobacter, the culture medium is a Yeast extract—casein hydrolysate—fatty acids (YCFA) medium. In some embodiments, the YCFA medium is modified.

In some embodiments, the YCFA medium comprises yeast extract in an amount of about 8 g/L, KH₂PO₄ in an amount of about 2 g/L, sodium propionate in an amount of about 1 g/L, cysteine in an amount of about 0.5 g/L, hemin in an amount of about 1 g/L, L-cysteine in an amount of about 1 g/L, and a vitamin solution as described below. In some embodiments, the modified YCFA medium further or instead comprises soy peptone in an amount ranging from more than 0 g/L to about 20 g/L, preferably from about 2 g/L to about 6 g/L, more preferably in an amount of about 4 g/L. In some embodiments, the modified YCFA medium comprises wheat peptone in an amount ranging from more than 0 g/L to about 20 g/L, preferably from about 2 g/L to about 6 g/L, more preferably in an amount of about 4 g/L. In some embodiments, the modified YCFA medium comprises Na₂CO₃ in an amount ranging from more than 0 g/L to about 20 g/L, preferably from about 2 g/L to about 6 g/L, more preferably in an amount of about 3 g/L. In some embodiments, the modified YCFA medium comprises MgCl₂ in an amount ranging from more than 0 mg/L to about 500 mg/L, preferably from about 25 mg/L to about 75 mg/L, more preferably in an amount of about 50 mg/L. In some embodiments, the modified YCFA medium comprises glutathione in an amount ranging from more than 0 g/L to about 5 g/L, preferably from about 0.5 g/L to about 1.5 g/L, more preferably in an amount of about 1 g/L. In some embodiments, the modified YCFA medium comprises ascorbate in an amount ranging from more than 0 g/L to about 1 g/L, preferably from about 0.25 g/L to about 0.75 g/L, more preferably in an amount of about 0.5 g/L. In some embodiments, the modified YCFA medium comprises soy peptone in an amount of about 4 g/L, wheat peptone in an amount of about 4 g/L, Na₂CO₃ in an amount of about 3 g/L, glutathione in an amount of about 1 g/L, ascorbate in an amount of about 1 g/L, ascorbate in an amount of about 0.5 g/L and.

In one embodiment, the modified YCFA medium comprises 8 g of yeast extract, 4 g of soy peptone, 4 g of wheat peptone, 5 g of KH₂PO₄, 3 g of Na₂CO₃, 50 mg of MgCl₂, 50 mg of CaCl₂), 1 mg of hemin, 1 mL Resazurin solution (1 g/L), 10 g of myo-inositol, 1 g of cysteine, 1 g of glutathion reduced, 0.5 g of ascorbate, 0.3 g of uric acid, 1 mL of vitamin solution and quantum satis 1000 mL of H₂O. In one embodiment, the vitamin solution used to prepare the modified YCFA medium comprises 2 mg of biotin, 2 mg of folic acid, 10 mg of pyridoxine-HCl, 2 mg of thiamine-HCl×2 H, 5 mg of riboflavin, 5 mg of nicotinic acid, 5 mg of D-Ca-pantothenate, 12 mg of vitamin B, 5 mg of p-aminobenzoic acid, 5 mg of lipoic acid and quantum satis 1000 mL of H₂O.

In some embodiments, the modified YCFA medium comprises soy peptone in an amount of about 4 g/L, wheat peptone in an amount of about 4 g/L, Na₂CO₃ in an amount of about 3 g/L, glutathione in an amount of about 1 g/L, ascorbate in an amount of about 1 g/L, ascorbate in an amount of about 0.5 g/L, MgCl₂ in an amount of 0.5 g/L, yeast extract in an amount if 8 g/L, KH₂PO₄ in an amount of 2 g/L, sodium propionate in an amount of 1 g/L, cysteine in an amount of 0.5 g/L, hemin in an amount of 1 g/L, L-cysteine in an amount of 1 g/L, and a vitamin solution as described hereinabove.

In one embodiment, the culture medium is free of living bacteria, i.e., the medium is sterilized. Means to sterilize a culture medium are known in the art and comprise, e.g., heating and irradiation. In another embodiment, the culture medium comprises living bacteria, preferably a substantially pure population of bacteria from the genus Dysosmobacter, and/or variant thereof.

In some embodiments, the culture medium is frozen and stored for future use, at a temperature of about −5°, −20° C., −70° C., −80° C. or −190° C.

In some embodiments, the culture medium is fermented. In some embodiments, the fermentation of the culture medium comprises the steps of:
1) incubating from about 10⁸ to about 10⁹ living bacteria from the genus Dysosmobacter, preferably from Dysosmobacter welbionis, more preferably from Dysosmobacter welbionis strain J115^{T}, in 50 mL of the modified YCFA medium for 48 hours at a temperature of 37° C. in anaerobic conditions;
2) incubating the culture of step 1) in 3.5 L of the modified YCFA medium for 48 hours at a temperature of 37° C. in anaerobic conditions;
3) collecting the culture medium from step 2), wherein the medium is fermented;
4) and optionally, removing the living bacteria from the collected medium of step 3) (i.e., sterilizing the medium).

In some embodiments, the extract from the bacteria from the genus Dysosmobacter, and/or variant thereof, or from the culture supernatant comprised in the composition for use according to the invention comprises at least one metabolite secreted by the bacteria.

In some embodiments, the composition for use according to the invention comprises metabolites produced and/or secreted by bacteria from the genus Dysosmobacter, preferably from Dysosmobacter welbionis, more preferably from Dysosmobacter welbionis strain J115^{T}.

In some embodiments, the at least one metabolite is selected from the group comprising or consisting of short chain fatty acids (SCFA) such as butyrate and acetate.

In some embodiments, the at least one metabolite is selected from the group comprising or consisting of butyrate, acetate, C8-3OH, C10-3OH, C12-3OH, C16-20H C16-3OH, C18-2OH C18-3OH, C18-130H, C18, C18:2 n-6, C22:2 n-6, C22:6 n-3, C18:1 n-9c, C22:1 n-9, C18:2 n-6, 10-TriHOME, 10-TriHOME, C12asn, C12asnGABAOH, 9,10-DiHOME, 12,13-DiHOME, 13-oxoODE, 8-HETE and 9-HODE. In some embodiments, the at least one metabolite is butyrate. In some embodiments, the at least one metabolite is acetate.

The Inventors have demonstrated that the effect of the bacteria from the genus Dysosmobacter, preferably from Dysosmobacter welbionis, more preferably from Dysosmobacter welbionis strain J115^{T}, is not limited to said metabolites produced and/or secreted by said bacteria. In particular, they have demonstrated that this effect is not limited to the presence or secretion of short chain fatty acids (SCFA) such as butyrate and acetate. Indeed, it appears from the results obtained by the Inventors that the culture medium obtained after fermentation of the bacteria totally inhibits the cell proliferation at 20% of concentration, thereby corresponding to a 10 to 20 times higher potency than the SCFA (see FIGS. 5B and 5D, SCFA-matched vs fermented).

In some embodiment, the composition for use according to the invention comprises an active agent from the supernatant of the culture medium of the bacteria from the genus Dysosmobacter, and/or variant thereof. In one embodiment, the active agent is a metabolite produced and/or secreted by the bacteria of the present invention as described hereinabove.

Another object of the present invention is a culture supernatant of the bacteria from the genus Dysosmobacter, and/or variants thereof, for use in preventing and/or treating breast cancer in a subject in need thereof.

In one embodiment, the culture supernatant is obtained by harvesting the culture medium of the bacteria from the genus Dysosmobacter, and/or variant thereof. In one embodiment, the culture supernatant is the fermented culture medium obtained after culture of the bacteria of the invention. In one embodiment, the culture of the bacteria from the genus Dysosmobacter, and/or variant thereof, is performed as disclosed hereinabove.

In a particular embodiment, the culture supernatant is obtained by the following steps:
1) incubating from about 10⁸ to about 10⁹ living bacteria from the genus Dysosmobacter, preferably from Dysosmobacter welbionis, more preferably from Dysosmobacter welbionis strain J115^{T}, in 50 mL of the modified YCFA medium for 48 hours at a temperature of 37° C. in anaerobic conditions;
2) incubating the culture of step 1) in 3.5 L of the modified YCFA medium for 48 hours at a temperature of 37° C. in anaerobic conditions;
3) collecting the culture medium from step 2), wherein the medium is fermented;
4) and optionally, removing the living bacteria from the collected medium of step 3).

The term “breast cancer” as used herein, refers to histologically or cytologically confirmed cancer of the breast. In some embodiments, the breast cancer is a carcinoma. In some embodiments, the breast cancer is an adenocarcinoma. In some embodiments, the breast cancer is a sarcoma.

In certain embodiment, the breast cancer is a hormone receptor-positive (HR+) breast cancer or a hormone receptor-negative (HR−) breast cancer. In one embodiment, the hormone receptor positive breast cancer is an estrogen receptor-positive (ER+) breast cancer and/or a progesterone receptor-positive (PR+) breast cancer. In some embodiments, the ER+breast cancer is luminal A breast cancer. In some embodiments, the ER+ breast cancer is luminal B breast cancer.

In some embodiments, the breast cancer is a human epidermal growth factor receptor 2-positive (HER2+) breast cancer. In some embodiments, the breast cancer is a human epidermal growth factor receptor 2-negative (HER2−) breast cancer.

In some embodiments, the breast cancer is a cancer with a combined expression of receptors, e.g., hormone receptor-positive and HER2 negative (HR+ HER2−).

In some embodiments, the breast cancer is a cancer of Group 1 (luminal A), Group 2 (luminal B), Group 3 (HER2+) or Group 4 (basal-like). Group 1 includes tumors that are ER+ and progesterone-receptor-positive (PR+) positive, but negative for HER2 (HR+ HER2−). Group 2 includes tumors that are ER+, PR− and HER2+. Group 3 includes tumors that are ER− and PR−, but HER2+. Group 4, which is also called TNBC, includes tumors that are ER−, PR− and HER2−.

In some embodiments, the breast cancer is a HER2+ breast cancer or a triple negative breast cancer (TNBC). In some embodiments, the breast cancer is HER2+ breast cancer, preferably HER2+ breast adenocarcinoma. In some embodiments, the breast cancer is a triple negative breast cancer (TNBC).

In some embodiments, the breast cancer is a non-invasive breast cancer, in particular a ductal carcinoma in situ or a lobular carcinoma in situ. In certain embodiment, the breast cancer is an invasive breast cancer, in particular selected in the group comprising or consisting of invasive ductal carcinoma, invasive lobular carcinoma, Paget's disease of the nipple, inflammatory breast cancer, Phyllodes tumor of the breast, locally advanced breast cancer and metastatic breast cancer.

In some embodiments, the type of tissue where breast cancer arises is milk ducts, milk-productive lobules or connective tissues.

In some embodiments, the breast cancer is a metastatic or a locally advanced breast cancer. The term “locally advanced breast cancer” refers to cancer that has spread from where it started in the breast to nearby tissue or lymph nodes, but not to other parts of the body.

In certain embodiments, the cancer is a metastatic cancer or a cancer susceptible to undergo metastasis, in particular a metastatic breast cancer or a breast cancer susceptible to undergo metastasis.

The term “metastatic breast cancer” refers to a cancer that has spread from the breast to other parts of the body, such as the bones, liver, lungs, or brain. Metastatic breast cancer may also be referred to as stage IV breast cancer. As used herein, the term “cancer susceptible to undergo metastasis” refers to an invasive cancer for which cancer cells may detach from the primary tumor and propagate in other organs, where they can grow to form secondary tumors, also referred to as metastases.

It is known in the art that a plethora of pathophysiological factors that can influence tumor growth such as, inter alia, conditions related to diet, obesity, glucose and lipid metabolism, and inflammation. These conditions are relevant within the scope of the present invention because of the ability of commensal bacteria, including bacteria from the genus Dysosmobacter, to influence these conditions.

In some embodiments, the subject further suffers from one or more disease or condition that accelerates tumor growth selected from the group comprising or consisting of obesity-related disorders, liver diseases, metabolic disorders, adipokine-related disorders and inflammatory diseases, and combinations thereof. In some embodiments, the one or more disease or condition that accelerates tumor growth is selected from the group comprising or consisting of obesity-related disorders, metabolic disorders, and inflammatory diseases, and combinations thereof. In some embodiments, the one or more disease or condition that accelerates tumor growth is selected from the group comprising or consisting of obesity-related disorders and metabolic disorders.

In some embodiments, the subject further suffers from obesity-related disorders. In some embodiments, the subject further suffers from liver diseases. In some embodiments, the subject further suffers from metabolic disorders. In some embodiments, the subject further suffers from adipokine-related disorders. In some embodiments, the subject further suffers from inflammatory diseases.

In some embodiment, the subject is not suffering from obesity-related disorders. In some embodiments, the subject has a normal BMI index.

In some embodiments, the subject further suffers from one or more disease or condition that accelerates tumor growth selected from the group comprising or consisting of obesity, non-alcoholic steatohepatitis (NASH), non-alcoholic fatty liver (NAFL), cirrhosis, diabetes mellitus, glucose intolerance, hyperglycemia, abnormal lipid metabolism, dyslipidemia, high cholesterol, elevated LDL-cholesterol, decreased HDL-cholesterol, elevated triglycerides and intestinal inflammation, and combinations thereof.

In some embodiments, the subject further suffers from obesity. In some embodiments, the subject further suffers from NASH. In some embodiments, the subject further suffers from NAFL. In some embodiments, the subject further suffers from cirrhosis. In some embodiments, the subject further suffers from diabetes mellitus. In some embodiments, the subject further suffers from glucose intolerance. In some embodiments, the subject further suffers from hyperglycemia. In some embodiments, the subject further suffers from abnormal lipid metabolism. In some embodiments, the subject further suffers from dyslipidemia. In some embodiments, the subject further suffers from high cholesterol. In some embodiments, the subject further suffers from elevated LDL-cholesterol. In some embodiments, the subject further suffers from decreased HDL-cholesterol. In some embodiments, the subject further suffers from elevated triglycerides. In some embodiments, the subject further suffers from intestinal inflammation.

In some embodiments, the subject suffers from gut microbiota imbalance. As used herein, gut microbiota imbalance refers to the overrepresentation or the underrepresentation of at least one microbial species, preferably at least one bacterial species. In some embodiments, the population of bacteria from the genus Dysosmobacter are underrepresented. In some embodiments, the population of Dysosmobacter welbionis is underrepresented. In some embodiments, the population of Dysosmobacter welbionis is decreased in subjects suffering from obesity and/or obesity-related disorders.

In some embodiments, the population of Dysosmobacter welbionis negatively correlates with the body mass index of the subject.

In some embodiments, the composition for use according to the invention comprises a therapeutically effective amount of said bacteria, preferably from about 1×10² to about 1×10¹⁵ CFU.

As used herein, “CFU” means “colony forming unit”.

In some embodiments, the composition for use according to the invention comprises a therapeutically effective amount of said bacteria, preferably from about 1×10⁴ to about 1×10¹² CFU, more preferably from about 1×10⁵ to about 1×10¹⁰ CFU, even more preferably from about 1×10⁶ to about 5×10⁹ CFU.

In some embodiments, the composition for use according to the invention comprises a therapeutically effective amount of said bacteria, preferably from about 1×10² to about 1×10¹⁵ CFU, from about 1×10⁴ to about 1×10¹² CFU, from about 1×10⁵ to about 1×10¹⁰ CFU, from about 1×10⁶ to about 5×10⁹ CFU. In some embodiments, the composition for use according to the invention comprises a therapeutically effective amount of said bacteria, preferably from about 1×10⁴ to about 1×10¹⁴ CFU, from about 1×10⁵ to about 1×10¹³ CFU, from about 1×10⁶ to about 1×10¹² CFU, from about 1×10⁷ to about 1×10¹¹ CFU, from about 1×10⁸ to about 1×10¹⁰ CFU, from about 2×10⁸ to about 6×10⁹ CFU.

In some embodiments, the composition for use according to the invention comprises a therapeutically effective amount of said bacteria, preferably from about 1×10⁴ to about 1×10¹² CFU/mL, more preferably from about 1×10⁵ to about 1×10¹⁰ CFU/mL, even more preferably from about 1×10⁶ to about 5×10⁹ CFU/mL.

In some embodiments, the composition for use according to the invention comprises a therapeutically effective amount of said bacteria, preferably from about 1×10⁴ to about 1×10¹⁴ CFU/mL, preferably from about 1×10⁵ to about 1×10¹³ CFU/mL, more preferably from about 1×10⁶ to about 1×10¹² CFU/mL, even more preferably from about 1×10⁷ to about 1×10¹¹ CFU/mL, 1×10⁸ to about 1×10¹⁰ CFU/mL, and even more preferably from about 2×10⁸ to about 6×10⁹ CFU/mL.

In some embodiments, the composition for use according to the invention comprises a therapeutically effective amount of said bacteria, preferably from about 1×10² to about 1×10¹⁵ CFU/g, preferably from about 1×10⁴ to about 1×10¹² CFU/g, more preferably from about 1×10⁵ to about 1×10¹⁰ CFU/g and even more preferably from about 1×10⁶ to about 5.10⁹ CFU/g. In some embodiments, the composition for use according to the invention comprises a therapeutically effective amount of said bacteria, preferably from about 1×10⁴ to about 1×10¹⁴ CFU/g, preferably from about 1×10⁵ to about 1×10¹³ CFU/g, more preferably from about 1×10⁶ to about 1×10¹² CFU/g, even more preferably from about 1×10⁷ to about 1×10¹¹ CFU/g, from about 1×10⁸ to about 1×10¹⁰ CFU/g, and even more preferably from about 2×10⁸ to about 6×10⁹ CFU/g.

In some embodiments, the composition for use according to the invention comprises a therapeutically effective amount of said bacteria, preferably from about 1×10⁶ to about 1×10¹⁰ CFU/g or CFU/mL, preferably from about 1×10⁸ to about 1×10¹⁰ CFU/g or CFU/mL, more preferably from about 1×10⁹ to about 1×10¹⁰ CFU/g or CFU/mL. In some embodiments, the composition for use according to the invention comprises a therapeutically effective amount of said bacteria, preferably from about 1×10⁶ to about 1×10¹¹ CFU/g or CFU/mL, preferably from about 1×10⁸ to about 1×10¹¹ CFU/g or CFU/mL, more preferably from about 1×10¹⁰ to about 1×10¹¹ CFU/g or CFU/mL.

In one embodiment of the invention, the composition for use according to the invention comprises an amount of fragment of the bacteria and/or variant thereof corresponding to an amount of bacteria and/or variant thereof ranging from about 1×10² to about 1×10¹⁵ cells/g, preferably from about 1×10⁴ to about 1×10¹² cells/g, more preferably from about 1×10⁵ to about 1×10¹⁰ cells/g and even more preferably from about 1×10⁶ to about 1×10⁹ cells/g. In one embodiment of the invention, the composition for use according to the invention comprises an amount of fragment of the bacteria and/or variant thereof corresponding to an amount of bacteria and/or variant thereof ranging from about 1×10⁴ to about 1×10¹⁴ cells/g, preferably from about 1×10⁵ to about 1×10¹³ cells/g, more preferably from about 1×10⁶ to about 1×10¹² cells/g, even more preferably from about 1×10⁷ to about 1×10¹¹ cells/g. In one embodiment of the invention, the composition for use according to the invention comprises an amount of fragment of the bacteria and/or variant thereof corresponding to an amount of bacteria and/or variant thereof ranging from about 1×10⁸ to about 1×10¹⁰ cells/g, preferably from about 1×10⁹ to about 1×10¹⁰ cells/g.

In one embodiment of the invention, the composition for use according to the invention comprises an amount of fragment of the bacteria and/or variant thereof corresponding to an amount of bacteria and/or variant thereof ranging from about 1×10² to about 1×10¹⁵ cells/mL, preferably from about 1×10⁴ to about 1×10¹² cells/mL, more preferably from about 1×10⁵ to about 1×10¹⁰ cells/mL and even more preferably from about 1×10⁶ to about 1×10⁹ cells/mL. In one embodiment of the invention, the composition for use according to the invention comprises an amount of fragment of the bacteria and/or variant thereof corresponding to an amount of bacteria and/or variant thereof ranging from about 1×10⁴ to about 1×10¹⁴ cells/mL, preferably from about 1×10⁵ to about 1×10¹³ cells/mL, more preferably from about 1×10⁶ to about 1×10¹² cells/mL, even more preferably from about 1×10⁷ to about 1×10¹¹ cells/mL. In one embodiment of the invention, the composition for use according to the invention comprises an amount of fragment of the bacteria and/or variant thereof corresponding to an amount of bacteria and/or variant thereof ranging from about 1×10⁸ to about 1×10¹⁰ cells/mL, preferably from about 1×10⁹ to about 1×10¹⁰ cells/mL.

In one embodiment of the invention, the composition for use according to the invention comprises an amount of fragment of the bacteria corresponding to an amount of bacteria ranging from about 1×10⁶ to about 1×10¹⁰ cells/g or cells/mL, preferably from about 1×10⁸ to about 1×10¹⁰ cells/g or cells/mL, more preferably from about 1×10⁹ to about 1×10¹⁰ cells/g or cells/mL.

In one embodiment of the invention, the composition for use according to the invention comprises an amount of fragment of the bacteria corresponding to an amount of bacteria ranging from about 1×10⁶ to about 1×10¹¹ cells/g or cells/mL, preferably from about 1×10⁸ to about 1×10¹¹ cells/g or cells/mL, more preferably from about 1×10¹⁰ to about 1×10¹¹ cells/g or cells/mL.

In some embodiments, the composition for use according to the invention is in the form of a pharmaceutical composition, further comprising a pharmaceutically acceptable carrier.

It is to be understood that the carrier must be “acceptable” in the sense of being compatible with the composition for use according to the invention, and not be deleterious upon being administered to an individual. Typically, the carrier does not produce an adverse, allergic or other untoward reaction when administered to an individual, preferably a human individual.

In some embodiments, the composition for use according to the invention or the pharmaceutical composition for use according to the invention further comprises at least another anticancer agent.

Anticancer agents are known from the state of the art. Non-limitative examples of anticancer agents include acalabrutinib, alectinib, alemtuzumab, anastrozole, avapritinib, avelumab, belinostat, bevacizumab, bleomycin, blinatumomab, bosutinib, brigatinib, carboplatin, carmustine, cetuximab, chlorambucil, cisplatin copanlisib, cytarabine, daunorubicin, decitabine, dexamethasone, docetaxel, doxorubicin, encorafenib, erdafitinib, etoposide, everolimus, exemestane, fludarabine, 5-fluorouracil, gemcitabine, ifosfamide, imatinib Mesylate, leuprolide, lomustine, mechlorethamine, melphalan, methotrexate, mitomycin, nelarabine, paclitaxel, pamidronate, panobinostat, pralatrexate, prednisolone, ofatumumab, rituximab, temozolomide, topotecan, tositumomab, trastuzumab, vandetanib, vincristine, vorinostat, zanubrutinib, and the likes.

In a preferred embodiment, the anticancer agent is an anticancer agent suitable to treat a breast cancer.

In certain embodiments, the anticancer agent is to be administered in combination with, concomitantly or sequentially, the composition for use according to the invention or the pharmaceutical composition for use according to the invention.

The present invention further relates to a medicament comprising the composition or pharmaceutical composition for use according to the invention. In some embodiments, said medicament is for treating and/or preventing breast cancer.

The present invention also relates to a composition comprising at least (a) bacteria from the genus Dysosmobacter, preferably from the species Dysosmobacter welbionis, more preferably from the strain Dysosmobacter welbionis J115^{T}, and/or variants and/or fragments thereof, and/or (b) a culture supernatant of bacteria from the genus Dysosmobacter, preferably from the species Dysosmobacter welbionis, more preferably from the strain Dysosmobacter welbionis J115^{T}, and/or variants thereof, for use for the manufacture of a medicament for treating and/or preventing breast cancer in a subject in need thereof.

Another object of the present invention is bacteria from the genus Dysosmobacter, preferably from the species Dysosmobacter welbionis, more preferably from the strain Dysosmobacter welbionis J115^{T}, and/or variants and/or fragments thereof, for use for the manufacture of a medicament for treating and/or preventing breast cancer in a subject in need thereof.

Still another object of the present invention is a culture supernatant of bacteria from the genus Dysosmobacter, preferably from the species Dysosmobacter welbionis, more preferably from the strain Dysosmobacter welbionis J115^{T}, and/or variants thereof, for use for the manufacture of a medicament for treating and/or preventing breast cancer in a subject in need thereof.

In some embodiments, the composition for use according to the invention is in the form of a nutraceutical or neutraceutical composition, further comprising a nutraceutically acceptable agent. In one embodiment, the nutraceutical composition comprises a nutraceutically effective amount of bacteria from the genus Dysosmobacter, and/or variants, extracts or fragments thereof.

In some embodiments, the nutraceutical composition is intended for ingestion by a human user, and may be useful for their preventative and medicinal qualities. In one embodiment, the nutraceutical composition of the invention is for use for preventing and/or treating breast cancer in a subject in need thereof.

In one embodiment, the nutraceutical composition is in form of a food or dietary supplement. In another embodiment, the nutraceutical composition is in form of a functional food. In some embodiments, the nutraceutical composition is formulated as a food additive, beverage, tablet, capsule, caplet, lozenge, syrup, suspension or emulsion.

In some embodiments, the nutraceutical composition further comprises one or more secondary nutraceutical components, wherein the secondary nutraceutical component is active nutritionally or therapeutically in the prevention and/or treatment of breast cancer. In some embodiments, the secondary nutraceutical component(s) is a vitamin, a mineral.

In some embodiments, the nutraceutically acceptable agent is selected from one or more of the group comprising talc, titanium dioxide, starch, cornstarch, modified cornstarch, kaolin, microcrystalline cellulose and powdered cellulose.

The present invention further relates to a prebiotic comprising one or more active ingredients or substances that increase the level of bacteria from the genus Dysosmobacter, preferably from the species Dysosmobacter welbionis, more preferably from the strain Dysosmobacter welbionis J115^{T}, and/or variants and/or fragments thereof, in the microbiota of a subject in need thereof.

In one embodiment, the prebiotic is for use in preventing and/or treating breast cancer in said subject.

In some embodiments, the prebiotic is selected from the group comprising or consisting of myo-inositol, inulin and inulin-type fructans, oligofructose, beta-glucans, xylose, arabinose, arabinoxylan, ribose, phytates, galactose, rhamnose, cellobiose, fructose, lactose, salicin, sucrose, glucose, esculin, tween 80, trehalose, maltose, mannose, mellibiose, mucus or mucins, raffinose, fructooligosaccharides, galacto-oligosaccharides, amino acids, alcohols, fermentable carbohydrates, water-soluble cellulose derivatives (e.g., methylcellulose, methyl ethyl cellulose, hydroxyethyl cellulose, ethyl hydroxyethyl cellulose, cationic hydroxyethyl cellulose, hydroxypropyl cellulose, hydroxyethyl methylcellulose, hydroxypropyl methylcellulose, and carboxymethyl cellulose), water-insoluble cellulose derivatives, unprocessed oatmeal, metamucil, all-bran, polyphenols and any combinations thereof.

The present invention also relates to a therapeutic combination product for its separate, simultaneous or sequential administration.

As used herein the term “therapeutic combination product” (that may also be referred to as a therapeutic kit of parts) refers to a product comprising or consisting of at least the 2 following parts: a first part comprising the composition for use, pharmaceutical composition for use or medicament according to the invention, and a second part comprising the prebiotic. In one embodiment, the therapeutic combination product is for treating and/or preventing breast cancer.

In some embodiments, the prebiotic is comprised in the composition for use, pharmaceutical composition for use or medicament according to the invention.

The present invention further relates to a composition comprising at least (a) one bacterium from the genus Dysosmobacter, preferably from the species Dysosmobacter welbionis, more preferably from the strain Dysosmobacter welbionis J115^{T}, and/or variants, extracts or fragments thereof, and/or (b) a culture supernatant of bacteria from the genus Dysosmobacter, preferably from the species Dysosmobacter welbionis, more preferably from the strain Dysosmobacter welbionis J115^{T}, and/or variants thereof, for use as an adjuvant to a treatment administered to a subject suffering from breast cancer.

As used herein, an “adjuvant” relates to an agent that potentiates the effect of a treatment, typically a breast cancer treatment. “Potentiates” means that the positive effects of the treatment on the disease or condition, preferably breast cancer, used with the adjuvant account for more than the sum of the effects of the treatment and the adjuvant taken individually.

In some embodiments, the breast cancer in the subject is treated by methods known in the art, e.g., hormonal therapy, chemotherapy and immunotherapy.

In some embodiments, the subject further suffers from one or more disease or condition that accelerates tumor growth selected from the group comprising or consisting of obesity-related disorders, liver diseases, metabolic disorders, adipokine-related disorders and inflammatory diseases, and combinations thereof. In some embodiments, the subject further suffers from one or more disease or condition that accelerates tumor growth selected from the group comprising or consisting of obesity, non-alcoholic steatohepatitis (NASH), non-alcoholic fatty liver (NAFL), cirrhosis, diabetes mellitus, glucose intolerance, hyperglycemia, abnormal lipid metabolism, dyslipidemia, high cholesterol, elevated LDL-cholesterol, decreased HDL-cholesterol, elevated triglycerides and intestinal inflammation, and combinations thereof.

In some embodiments, the subject suffers from an imbalance of gut microbiota. In some embodiments, the gut microbiota of the subject comprises a decreased population of bacteria from the genus Dysosmobacter, preferably Dysosmobacter welbionis, compared to a healthy subject.

The present invention further relates to a method of treating and/or preventing breast cancer in a subject in need thereof, comprising administering to the subject a therapeutically effective amount of a composition comprising at least (a) bacteria from the genus Dysosmobacter, preferably from the species Dysosmobacter welbionis, more preferably from the strain Dysosmobacter welbionis J115^{T}, and/or variants, extracts or fragments thereof, and/or (b) a culture supernatant of bacteria from the genus Dysosmobacter, preferably from the species Dysosmobacter welbionis, more preferably from the strain Dysosmobacter welbionis J115^{T}, and/or variants thereof.

Therapeutically effective amounts of the composition have been described hereinabove.

In some embodiments, the method further comprises administering to the subject a therapeutically effective amount of another anticancer agent. Examples of other anticancer agents have been described hereinabove. Thus, the present invention also relates to a method of treating and/or preventing breast cancer in a subject in need thereof, comprising administering to the subject a therapeutically effective amount of a pharmaceutical composition comprising (i) a composition comprising bacteria from the genus Dysosmobacter, and/or variants, extracts or fragments thereof, and (ii) another therapeutic agent, wherein the other therapeutic agent is an anticancer agent.

In one embodiment, the method is used when the subject suffers from breast cancer, and aims at slowing down or reversing the progression of tumor growth. In another embodiment, the method is used when the subject does not suffer from breast cancer, and aims at preventing the appearance of breast cancer. In one embodiment, the method is used acutely. In another embodiment, the method is used chronically.

In some embodiments, the subject further suffers from one or more disease or condition that accelerates tumor growth selected from the group comprising or consisting of obesity-related disorders, liver diseases, metabolic disorders, adipokine-related disorders and inflammatory diseases, and combinations thereof. In some embodiments, the subject further suffers from one or more disease or condition that accelerates tumor growth selected from the group comprising or consisting of obesity, non-alcoholic steatohepatitis (NASH), non-alcoholic fatty liver (NAFL), cirrhosis, diabetes mellitus, glucose intolerance, hyperglycemia, abnormal lipid metabolism, dyslipidemia, high cholesterol, elevated LDL-cholesterol, decreased HDL-cholesterol, elevated triglycerides and intestinal inflammation, and combinations thereof.

In some embodiments, the subject suffers from an imbalance of gut microbiota. In some embodiments, the gut microbiota of the subject comprises a decreased population of bacteria from the genus Dysosmobacter, preferably Dysosmobacter welbionis, compared to a healthy subject.

The present invention further relates to a composition comprising at least (a) bacteria from the genus Dysosmobacter, preferably from the species Dysosmobacter welbionis, more preferably from the strain Dysosmobacter welbionis J115^{T}, and/or variants thereof, and/or (b) a culture supernatant of bacteria from the genus Dysosmobacter, preferably from the species Dysosmobacter welbionis, more preferably from the strain Dysosmobacter welbionis J115^{T}, and/or variants thereof, for use as a probiotic. In certain embodiments, the bacteria comprised in the composition are the strain J115^{T}, and/or variant thereof, which is beneficial for improving the gastrointestinal environment of a subject.

The present invention also relates to a method for increasing the population of bacteria from the genus Dysosmobacter, preferably Dysosmobacter welbionis, more preferably Dysosmobacter welbionis strain J115^{T}, within the gut microbiota of a subject, comprising administering the bacteria to the subject, wherein the bacteria is comprised in a composition, pharmaceutical composition, or a prebiotic. In a preferred embodiment, the subject is at risk of developing breast cancer, or suffers from breast cancer.

The present invention also relates to a method for treating gut microbiota imbalance, comprising administering to a subject a composition or a prebiotic comprising at least (a) bacteria from the genus Dysosmobacter, preferably Dysosmobacter welbionis, more preferably Dysosmobacter welbionis strain J115^{T}, and/or (b) a culture supernatant of bacteria from the genus Dysosmobacter, preferably from the species Dysosmobacter welbionis, more preferably from the strain Dysosmobacter welbionis J115^{T}, and/or variants thereof.

### BRIEF DESCRIPTION OF THE DRAWINGS

FIG. 1A-1G is set of graphs showing that Dysosmobacter welbionis J115^{T} partly alleviates high-fat diet driven tumor growth. FIG. 1A shows bodyweight evolution of under normal diet (ND) or high-fat diet (HFD). Arrow indicates the induction of tumors. FIG. 1B shows E0771 tumor volume evolution. FIG. 1C shows tumor volume evolution with focus on ND and HFD groups. FIG. 1D shows tumor volume evolution with focus on HFD groups treated or not with J115^{T}. FIG. 1E shows survival curve deduced by calculating the time needed for each tumor to reach 300 mm³ from linear regressions. FIG. 1F shows survival curve focused on the difference between ND and HFD groups. FIG. 1G shows survival curve focused on the difference between HFD and HFD J115^{T} groups. Sample size: n=9 for ND, n=8 for HFD and n=11 for HFD J115^{T}. Mice harboring tumors smaller than 100 mm³ were excluded. Statistical analysis: (FIG. 1A, 1B) Two-way ANOVA analysis followed by Tukey's multiple comparison test. ***p<0.001 (HFD vs ND) and $ p<0.05 (HFD J115^{T} vs HFD). (FIG. 1C, 1D) Two-way ANOVA analysis followed by Sidak's multiple comparison test. *p<0.05; ***p<0.001. (FIG. 1E, 1F, 1G) Log-rank (Mantel-Cox) test. *p<0.05.

FIG. 2A-2E is set of graphs showing that Dysosmobacter welbionis J115^{T} alleviates high-fat diet driven tumor growth and confirms the results of FIG. 1. FIG. 2A shows bodyweight evolution of under normal diet (ND) or high-fat diet (HFD). Arrow indicates the induction of tumors. FIG. 2B shows E0771 tumor volume evolution. FIG. 2C shows survival curve deduced by calculating the time needed for each tumor to reach 400 mm³ from linear regressions. FIG. 2D shows survival curve focused on the difference between ND and HFD groups. FIG. 2E shows survival curve focused on the difference between HFD and HFD J115^{T} groups. Sample size: n=9 for ND, n=10 for HFD and n=8 for HFD J115^{T}. Mice harboring tumors smaller than 100 mm³ and showing less than 50% growth (compared to day 6) were excluded. Statistical analysis: (FIG. 2A, 2B) Two-way ANOVA analysis followed by Tukey's multiple comparison test. *p<0.05; **p<0.01; ***p<0.001 (HFD vs ND) and $$ p<0.01 (HFD J115^{T} vs HFD). (FIG. 2C, 2D, 2E) Log-rank (Mantel-Cox) test. *p<0.05.

FIG. 3A-3B is set of graphs showing that Dysosmobacter welbionis J115^{T} alleviates high-fat diet driven tumor growth and confirms the results of FIGS. 1 and 2. FIG. 3A shows bodyweight evolution of under high-fat diet (HFD). Arrow indicates the induction of tumors. FIG. 3B shows E0771 tumor volume evolution. Statistical analysis: Two-way ANOVA followed by Sidak's multiple comparison test. **p<0.01.

FIG. 4A-4B is set of graphs showing that Dysosmobacter welbionis J115 alleviates tumor growth in a non-obesity related context. FIG. 4A shows bodyweight evolution of Balb/c mice under normal diet (ND). FIG. 4B shows 4^{T}1 tumor volume evolution. Statistical analysis: Two-way ANOVA followed by Sidak's multiple comparison test. **p<0.01.

FIG. 5A-5D is a set of histograms showing that Dysosmobacter welbionis J115^{T} supernatant decreases cell density and proliferation in triple-negative breast cancer (TNBC) models in vitro. FIG. 5A-5B show cell density (A) and cell proliferation (B) of PY8119 cells after 24 h of treatment with either non-fermented bacterial medium, fermented bacterial medium and pH and short-chain fatty acid (SCFA) content-matched (to fermented) phosphate buffered solution (PBS) at indicated concentrations. FIG. 5C-5D show cell density (C) and cell proliferation (D) of E0771 cells after 24 h of treatment with either non-fermented bacterial medium, fermented bacterial medium or pH and short-chain fatty acid (SCFA) content-matched (to fermented medium) phosphate buffered solution (PBS) at indicated concentrations. The pH of the fermented medium is 6.1 and SCFA content is equal to 25 mM of butyrate and 60 mM of acetate. All data are expressed as mean±SEM of three independent experiments. Statistical analysis: Nested One-way ANOVA analysis followed by Dunnett's multiple comparison test. *p<0.05; **p<0.01; ***p<0.001.

### EXAMPLES

The present invention is further illustrated by the following example.

### Example 1

### Materials and Methods

### Bacteria Culture

Dysosmobacter welbionis J115^{T} was cultured anaerobically in a modified YCFA medium supplemented with 10 g/L inositol. Cultures were centrifuged at 5000 g during 15 min at 4° C. and the supernatant was removed. cells were resuspended in anaerobic PBS-carbonate buffer supplemented with 15% (vol/vol) trehalose then immediately frozen in anaerobic vials and stored at −80° C. The number of total and cultivable bacteria administered to the mice was calculated by plating the bacterial culture before centrifugation and the bacterial suspension after freezing for mice administration.

### Cell Culture

E0771 and PY8119 Mus musculus mammary gland adenocarcinoma cell lines were acquired from American Type Cell Culture (ATCC) and stored according to the supplier's instructions. 4T1 cells were derived from the mammary gland of BALB/c mouse strain. E0771 and 4T1 cells were maintained in Dulbecco's Modified Eagle Medium (GIBCO, Thermo Fisher Scientific) with 25 mM glucose, 4 mM glutamine, and 25 mM HEPES, supplemented with 10% heat-inactivated Fetal Bovine Serum (FBS) (Thermo Fisher Scientific). PY8119 cells were maintained in culture in F-12K medium (GIBCO, Thermo Fisher Scientific) with 7 mM glucose and 2 mM glutamine, supplemented with 5% heat-inactivated FBS (Thermo Fisher Scientific). They were cultured in a humidified atmosphere at 37° C. and 5% CO₂.

### Cell Density

Cell density was assessed using the PrestoBlue reagent (ThermoFisher Scientific) according to the manufacturer's instruction. Briefly, cells were treated for 24 hours with non-fermented or fermented YCFA medium described above. To discriminate for short-chain fatty acid's contribution and the effect of pH, phosphate buffered solution (PBS) containing 25 mM of butyrate and 60 mM of acetate at pH 6.1 was used. PrestoBlue Reagent was added at a concentration of 10% in each well. After 2 hours of incubation, fluorescence intensity was measured (λex/λem=560/590 nm) using a plate reader (SpectraMax M2e, Molecular Devices). All data were normalized to the fluorescence intensity of untreated wells and expressed in percent of control.

### Cell Proliferation

Cell proliferation was measured by using a 5-bromo-2-deoxyuridine (BrDu) incorporation ELISA-based kit (Roche) following the provider's protocol. After 24 hours of incubation with indicated treatments described above, BrDu was added to the culture medium for 2 hours. Then, after a fixation step of the cells with a solution provided by the manufacturer and binding of anti-BrDu antibody coupled to a peroxidase, cellular proliferation was assessed by measuring the absorbance at 370 nm using a plate reader (SpectraMax M2e, Molecular Devices). All data were normalized to the absorbance of untreated wells and expressed in percent of control.

### Mice

All mouse experiments were approved by the ethical committee for animal care of the Health Sector of the Université Catholique de Louvain, under the specific number 2021/UCL/MD/04 and its amendment 2022/UCL/MD/A10 and performed in accordance with the guidelines of the local ethics committee and in accordance with the Belgian Law of 29 May 2013, regarding the protection of laboratory animals (agreement number LA1230467). Specific pathogen-free (SPF) certified 8-week-old female C57BL/6JRj mice (Janvier Labs) were used for the experiments. Cages were randomly assigned to experimental groups to ensure that each group was matched in terms of bodyweight at the beginning of normal diet (ND) (D10012M; Research Diets) and high-fat diet (HFD) 60% kcal (D12492; Research Diets) feeding and before tumor induction. Bodyweight was assessed weekly.

### Oral Bacteria Administration

Freshly thawed Dysosmobacter welbionis J115^{T} was administered daily in late post-prandial stage by oral gavage. Each mouse received at least 1.0×10⁹ live (cultivable) bacteria in 0.2 mL. ND and HFD control groups were given an oral gavage of an equivalent volume of PBS-carbonate buffer supplemented with 15% (weight/vol) trehalose.

### Tumor Growth Experiments

E0771 tumors were induced by subcutaneous injection of 1×10⁶ cells in the fifth mammary fat pad of C57Bl/6JRj female mice 6 weeks after the beginning of the HFD treatment. 4T1 tumors were induced by subcutaneous injection of 2×10⁵ cells in the fifth mammary fat pad of 8 weeks old BALB/c mice. E0771 cells were freshly passaged just before the injection and prepared as a mixture of 1:1 PBS and Matrigel (Corning) and injected within 30 min. 4T1 cells were freshly passaged just before the injection and prepared in a PBS solution and injected within 30 min. Tumor size was monitored at least twice a week (preliminary experiment) or every day (confirmation) and measured using an electronic caliper in a simple blind manner.

### Statistical Analysis

All statistical analyses were performed using GraphPad Prism software version 9.1.2.

### Results

In the present invention, Dysosmobacter welbionis J115^{T} was tested for its ability to reduce or delay the development of cancer in obese conditions in vivo and in vitro.

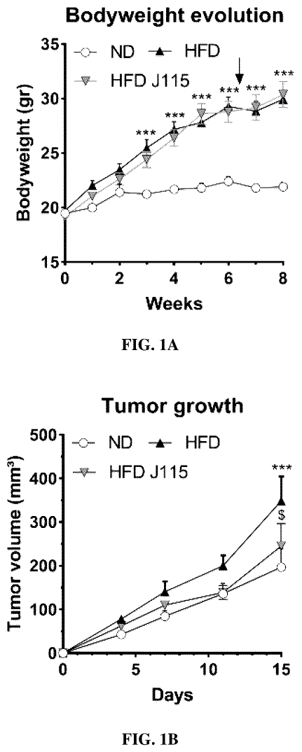

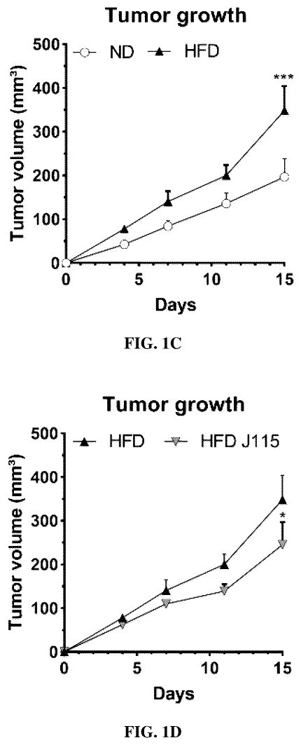

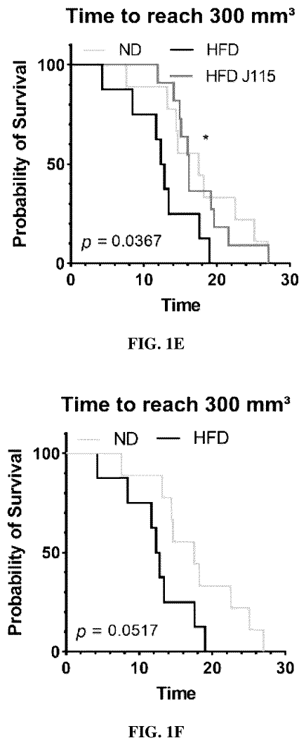

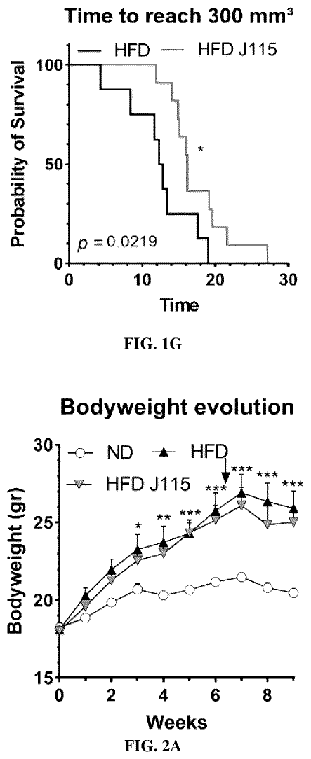

More precisely, as a first model, triple negative breast cancer cells were used. Obesity was induced in mice by using a high-fat diet treatment, then the TNBC cells were injected in the mammary gland of mice (FIG. 1A, see arrow=injection of tumor cells). Mice were treated daily with either a placebo or Dysosmobacter welbionis J115^{T}. At day 15, obese mice treated with the placebo exhibited a significantly higher tumor growth with an 80% bigger tumor volume than the lean mice (FIGS. 1B and 1C), whereas obese mice treated with Dysosmobacter welbionis J115^{T} displayed a significantly reduced development of the tumor which was similar to the lean mice (FIGS. 1B and 1D).

Obese mice are also characterized by a faster tumor growth as shown in FIGS. 1E and 1F, the time to reach the same volume of tumor is shortened in obese mice versus lean mice, in other words, the tumor of the lean mice will reach a volume of 300 mm³ in 27 days when the obese mice have reached this size in only 19 days (FIGS. 1E and 1F). Altogether, this effect on the delayed tumor size induced by Dysosmobacter welbionis J115^{T} strongly extended the time required to reach the same volume as the one observed in the obese mice. Hence, Dysosmobacter welbionis J115^{T} treated mice will reach a volume of 300 mm³ in 27 days, which is similar to the lean mice and much delayed than the obesity (weight) matched mice receiving the placebo (FIGS. 1E and 1G). Surprisingly, this beneficial effect was not linked to a body weight loss of the Dysosmobacter welbionis J115^{T} treated mice (FIG. 1A).

This first set of data convincingly show that a non-invasive approach such as using Dysosmobacter welbionis J115^{T} could be linked to such a strong delay in cancer development and that surprisingly this is not due to the anti-obesity effect of Dysosmobacter welbionis J115^{T}.

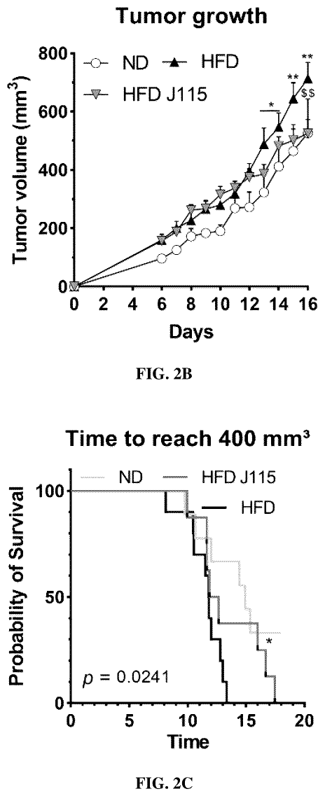

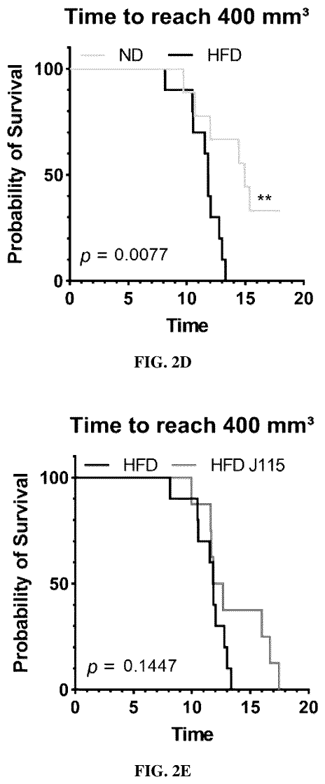

In a second set of independent experiment (FIG. 2), the same beneficial effects of Dysosmobacter welbionis J115^{T} on the reduction of tumor growth were completely replicated (FIGS. 2B and 2C) and this effect remained significantly present even if the volume of 400 mm³ was selected (FIGS. 2D and 2E) instead of 300 mm³ as threshold.

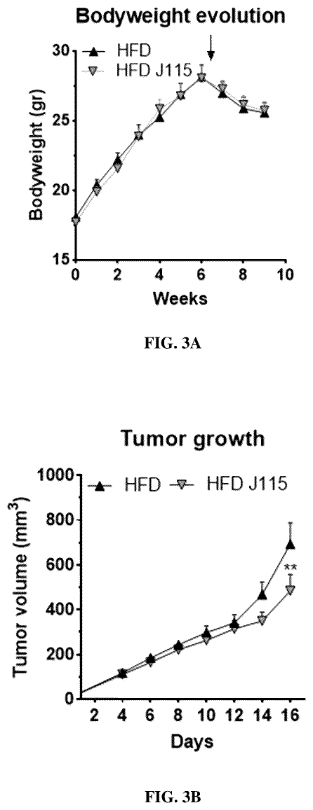

Finally, a third set of independent experiment (FIG. 3) confirmed the beneficial effects of Dysosmobacter welbionis J115^{T} on the reduction of tumor growth in a obesogenic context (FIG. 3B).

In the present invention, Dysosmobacter welbionis J115^{T} was also tested for its ability to reduce or delay the development of cancer in non-obesogenic conditions in vivo.

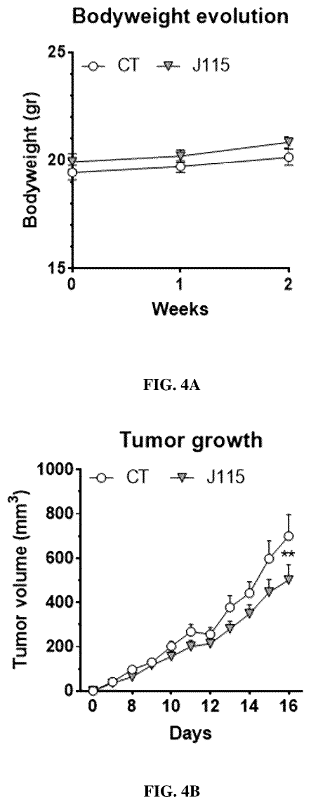

Cells were subcutaneously injected in the fifth mammary fat pad of mice (FIG. 4A). Mice were then treated daily with either a placebo or Dysosmobacter welbionis J115^{T}. At day 16, mice treated with Dysosmobacter welbionis J115^{T} displayed a significantly reduced growth of the tumor compared to the untreated mice (FIG. 4B). These experiments show that Dysosmobacter welbionis J115^{T} delays cancer development independently of obesity.

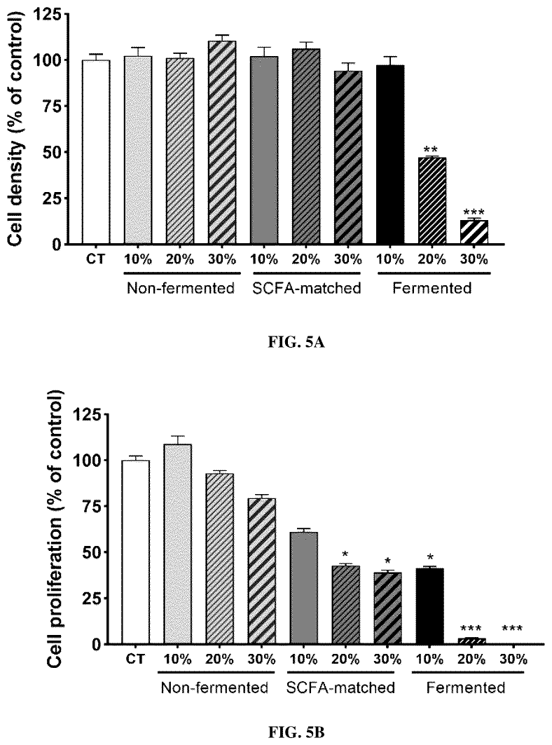

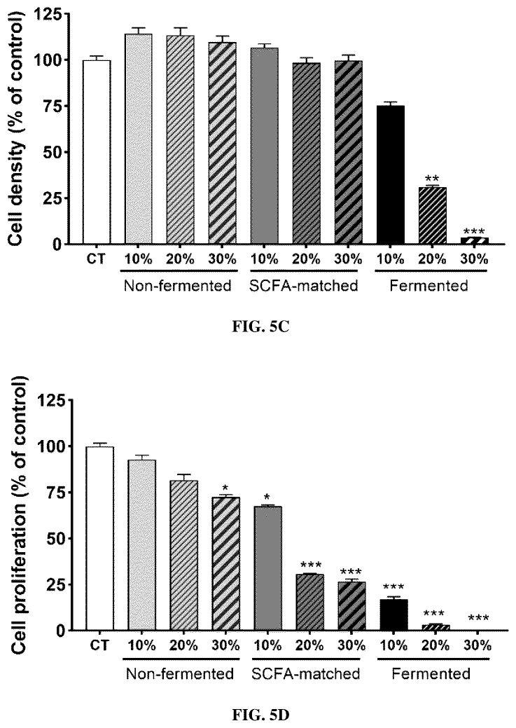

Next, the effects of the potential metabolites produced by Dysosmobacter welbionis J115^{T} were investigated by using in vitro testing with the fermented culture medium with Dysosmobacter welbionis J115^{T} (FIG. 5). Fermented culture medium with Dysosmobacter welbionis J115^{T} significantly reduces cell density of the two different models of triple-negative breast cancer (E0771 and PY8119 cells) in a dose dependent manner after 24 h of treatment (FIGS. 5A and 5C). Conversely, the non-fermented bacterial medium was not affecting this parameter.

Because Dysosmobacter welbionis J115^{T} produces short chain fatty acids SCFA (butyrate, acetate), the non-fermented bacterial medium was tested, adjusted for the pH and supplemented with SCFA which is therefore a content-matched (to fermented). Neither the non-fermented nor the non-fermented medium enriched in SCFA could affect the cell density (FIG. 5, SCFA-matched).

The fermented culture medium with Dysosmobacter welbionis J115^{T} decreases cell proliferation of both E0771 and PY8119 cells after 24 h of treatment, whereas the non-fermented medium has no effect on this parameter. However, cell proliferation was reduced in SCFA-matched medium but without reaching the same levels as the one obtained with Dysosmobacter welbionis J115^{T}. Indeed, the medium coming from Dysosmobacter welbionis J115^{T} fermentation was totally inhibiting the cell proliferation already at 20% of concentration, thereby corresponding to a 10 to 20 times higher potency than the SCFA (FIGS. 5B and 5D, SCFA-matched vs fermented).

These data show that an effect of specific metabolites produced by Dysosmobacter welbionis J115^{T} and different than SCFA is mediated the anti-tumoral effects of the bacteria.

1-15. (canceled)

16. An in vitro method for obtaining clinical-grade CD8⁺ CD45RC^{low/-} Method of treating and/or preventing breast cancer in a subject in need thereof, comprising administering to the subject a therapeutically effective amount of a composition comprising at least (a) bacteria from the genus Dysosmobacter, and/or variants, and/or extracts and/or fragments thereof, and/or (b) a culture supernatant of bacteria from the genus Dysosmobacter, and/or variants thereof.

17. The method according to claim 16, wherein said bacteria belong to the species Dysosmobacter welbionis.

18. The method according to claim 16, wherein said bacteria belong to the strain J115^{T}, deposited at the BCCM/LMG on Mar. 14, 2018 as LMG P-30603.

19. The method according to claim 16, wherein said breast cancer comprises at least one mutation on the gene encoding a receptor selected from the group consisting of Progesterone Receptor (PR), Estrogen Receptor (ER), and Human Epidermal Growth Factor Receptor-2 (HER2).

20. The method according to claim 16, wherein said breast cancer is selected from the group consisting of luminal A breast cancer, luminal B breast cancer, HER2-positive breast cancer, and triple negative breast cancer (TNBC).

21. The method according to claim 16, wherein said breast cancer is triple negative breast cancer.

22. The method according to claim 16, wherein said subject further suffers from one or more disease or condition that accelerates tumor growth selected from the group consisting of obesity-related disorders, liver diseases, metabolic disorders, adipokine-related disorders and inflammatory diseases, and combinations thereof.

23. The method according to claim 16, wherein said subject further suffers from one or more disease or condition that accelerates tumor growth selected from the group consisting of obesity, non-alcoholic steatohepatitis (NASH), non-alcoholic fatty liver (NAFL), cirrhosis, diabetes mellitus, glucose intolerance, hyperglycemia, abnormal lipid metabolism, dyslipidemia, high cholesterol, elevated LDL-cholesterol, decreased HDL-cholesterol, elevated triglycerides and intestinal inflammation, and combinations thereof.

24. The method according to claim 16, wherein said composition comprises a therapeutically effective amount of said bacteria, from about 1×10² to about 1×10¹⁵ CFU.

25. The method according to claim 16, wherein said composition comprises live bacteria.

26. The method according to claim 16, wherein said composition comprises dead or killed bacteria.

27. The method according to claim 16, wherein said composition comprises pasteurized bacteria.

28. The method according to claim 16, wherein said composition further comprises at least another anticancer agent.

29. The method according to claim 16, wherein said composition is in the form of a pharmaceutical composition further comprising a pharmaceutically acceptable carrier.

30. A method of preventing and/or treating breast cancer in a subject in need thereof, comprising administering to the subject a prebiotic comprising one or more active ingredients or substances that increase the level of bacteria from the genus Dysosmobacter in the microbiota of said subject.

31. A method for treating breast cancer in a subject in need thereof comprising administering to the subject an adjuvant to a treatment consisting of a composition comprising at least (a) one bacterium from the genus Dysosmobacter, and/or variants, extracts or fragments thereof, and/or (b) culture supernatant of one bacterium from the genus Dysosmobacter, and/or variants thereof.
